(19) United States
(12) Patent Application Publication
(10) Pub. No.: US 2022/0152401 A1
(43) Pub. Date: May 19, 2022

(54) Biological Battery

(71) Applicant: Austin James Georgiades, Marlborough, CT (US)

(72) Inventor: Austin James Georgiades, Marlborough, CT (US)

(21) Appl. No.: 17/069,867

(22) Filed: Nov. 16, 2020

(51) Int. Cl. A61N 1/378; A61N 1/36; C12N 11/04

(52) U.S. Cl. CPC A61N 1/3785; C12N 11/04; A61N 1/36003

(57) ABSTRACT

A biological battery consists of uniformly-polarized biological cells capable of generating an electrochemical gradient compartmentalized into single layers which are separated from each other by sections of membrane capable of converting the energy contained within an electrochemical gradient into electrical current. This arrangement of stacked cell layers is encapsulated by an ultrafiltration membrane permeable to monosaccharides and amino acids but not large proteins. This enables the biological cells contained within to survive and generate the electrochemical gradients needed for power, but prevents an immune response against them by the organism it is implanted in. Biological batteries produce electrical energy capable of powering a circuit from an organism's own bodily chemical energy, thus eliminating the need for external power sources to power an implanted electronic device.

The basic function of a biological battery is to harness electrochemical gradients produced by cells in vivo to generate usable current within a circuit. This patent describes a mechanism by which chemical energy present in the body can be transformed into usable electrical energy, thus allowing power for implanted devices to be generated internally, without a need for external power.

The exterior of the battery consists an enveloping, biocompatible, ultrafiltration membrane (Membrane Type 1) permeable to small molecules like glucose and amino acids, but impermeable to larger proteins like antibodies (such as WO2008086477B1). Biological cells contained within the battery are therefore able to access the necessary building blocks needed to construct and maintain cell machinery and to power internal cellular processes (which are naturally abundant within the body) without inducing an immune response.

The biological cells enclosed within Membrane Type 1 must be capable of pumping ions to form an electrochemical gradient. All biological cells within the battery are oriented so they pump ions in the same direction, meaning polarization must be induced after the cells are placed in their respective compartments within the battery. A good candidate cell to do this would be the acid-producing cells of the stomach (also called parietal or oxyntic cells). These epithelial cells naturally polarize so that they pump acidic protons (H⁺) out one side into the stomach lumen and alkaline bicarbonate (HCO₃⁻) out the opposite side into the blood. In the case that oxyntic cells were used in a biological battery, this would mean they must all be oriented so that protons are pumped in one direction and bicarbonate is pumped in the opposite direction. The oxyntic cells form a sheet that is one biological cell thick (an oxyntic cell layer or OCL) with cells within the OCL sharing a direction of polarization. Batteries can contain multiple OCLs, in which case, all OCLs share a direction of polarization as well.

Separating these OCLs would be sections of a membrane (Membrane Type 2) capable of converting an electrochemical gradient of a conjugate acid-base pair into electrical energy (such as U.S. Pat. No. 4,311,771A). The basic mechanism of operation of Membrane Type 2 is to accept an electron from the negatively-charged anion onto its surface, which must traverse a circuit before being able to associate with a cation on the other side of the membrane, in a way very similar to a fuel cell. In this case, the cation is the electron acceptor, and the anion is the electron donor. The conjugate-base anion, once losing its electron and becoming neutral, is then able to diffuse through Membrane Type 2 and recombine with the conjugate acid cation, which has accepted the donated electron that has traversed the circuit to the other side of Membrane Type 2. The uniform orientation of all OCLs would mean that all sections of Membrane Type 2 would have a high gradient of protons (conjugate acid) and bicarbonate (conjugate base) across them. So, while Membrane Type 1 serves to separate the OCLs from the outside environment, Membrane Type 2 serves to internally separate OCLs from each other and forms the basis of electricity generation needed to power a circuit.

Two OCLs separated by sections of Membrane Type 2 form an electrochemical cell. Batteries can be formed by stacking electrochemical cells together. Within an electrochemical cell, conjugate-acid cations from one OCL abut the negative, conjugate-base ions of a neighboring OCL, separated by a section of Membrane Type 2. It is here that the electrochemical gradient is harnessed for the creation of electrical energy. The number of electrochemical cells is determined by the number of sections of Membrane Type 2 that are present in the battery, with the number of possible Membrane Type 2 sections being one less than the number of OCLs (unless the battery is circular, in which case the number of Membrane Type 2 sections is equal to the number of OCLs). The reason all biological cells within a biological battery must share the same direction of polarization is to ensure that a conjugate acid-base gradient is formed across every section of Membrane Type 2. If any OCL had a different direction of polarization than its adjacent OCLs, the necessary electrochemical gradients needed to generate electrical current could not form across the sections of Membrane Type 2 that frame it and that section of the battery would be essentially useless.

It should be noted that all sections of Membrane Type 2 are fused along their entire periphery to the encapsulating Membrane Type 1, which forms an isolated compartment for each OCL, meaning the electrochemical gradients formed across Membrane Type 2 must neutralize by passing through Membrane Type 2, not by passing through an open gap in the membrane separating adjacent OCL chambers. This is needed to optimize the efficiency of current generation in the battery. In addition, Membrane Type 1 is fused at all edges to form a continuous layer that separates OCLs from the outside cellular environment. This is necessary to ensure that no macromolecules like antibodies can interact with the oxyntic cells in induce an immune response as a result their presence (these biological cells would almost certainly not be native to the body of someone with a biological battery inside of them and thus would induce an immune response).

Biocompatible leads from each section of Membrane Type 2 would pass through Membrane Type 1 and be collected together into positive and negative electrical contacts that supply power to attached circuitry. In this way, the electrical energy supplied by the biological battery can be used to power a circuit when implanted into a living organism using only the chemical energy naturally present in the body. OCLs can be unidirectionally polarized by either blotting proteins that induce oxyntic cell polarization onto specific sides of Membrane Type 2 before oxyntic cells are added to compartments, or by using an outside power source to create a voltage across OCL compartments to induce cell polarization in response to electric potential. It should be noted that other cell types besides oxyntic cells could be used to generate a conjugate acid-base gradient capable of powering a biological battery.

Biological batteries are the ultimate solution to supplying implants with electrical energy. Though it is possible to use biological batteries in an industrial capacity, to, say, generate meaningful amounts of electrical energy from partially-processed biomass (such as sewage), it is uniquely suited to the task of powering the circuitry of implanted devices. Preliminary, conservative calculations of the theoretical energy output of a biological battery containing 11,000 oxyntic cells at 3 watts. This assumes no ion leakage through Membrane Type 1, so cut the power supplied to 25% and roughly 50,000 oxyntic cells are needed to produce three watts. This also assumes all cells within the battery would have enough glucose available to function at peak capacity. Considering glucose would become scarcer towards the center of the battery, cut the power output by a factor of 100. This means it would take 5 million oxyntic cells to produce a 3-watt biological battery, with 50 million capable of an output of 30 watts. Considering the average epithelial cell is about 20 microns in diameter, this means the total volume of oxyntic cells required to achieve this would have a volume less than 1 cubic millimeter. Assuming those cells are put into a battery with 5 OCLs, if the profile of the battery was square, then each side would be 6.3 cm in length. A battery with an output of 100 watts, if 10 OCLs thick and square, would have side lengths of 8.1 cm. Thus, even by conservative estimates, biological batteries look to be incredibly powerful sources of energy for implanted circuitry.

This would enable the construction of much more complex implants that demand the energy that only a biological battery can provide. Pacemakers can be made more powerful, perhaps bone screws and plates can have integrated force sensors that could then be wirelessly transmitted to an outside receiver. It would be possible to power a brain-computer interface (BCI) all day and night using the energy of the body alone. Electrodes arrays placed into the muscles of those with paralysis and powered with biological batteries could receive communications from an implanted BCI telling them to stimulate muscle activity in certain muscle groups at certain times in a particular way. Using biofeedback, a paralyzed person could learn to walk again and spinal injuries could be effectively bypassed. Cochlear implants could be powered internally, seamlessly integrated into the body, out of sight and out of mind, just powering essential circuitry without ever needing a second thought. Bionic eyes and biometric implants, the list goes on, could all be implanted with a long-lasting, constant, internal source of power using a biological battery.

### DRAWING

FIG. 1:

Here is displayed the outside of a linear biological battery. This is a membrane (Membrane Type 1) permeable to small molecules like carbohydrates and amino acids, but impermeable to proteins (such as immunoglobulins). The arrows on all sides indicate that the battery can be scaled in any dimension (or any combination of dimensions) to accommodate almost any desired geometry for various applications. In addition, the positive and negative leads sticking out of the battery can be modified to accommodate various applications as well. Leads can be connected in any combination of parallel or series to produce the desired output current and voltage needed for a specific application.

FIG. 2:

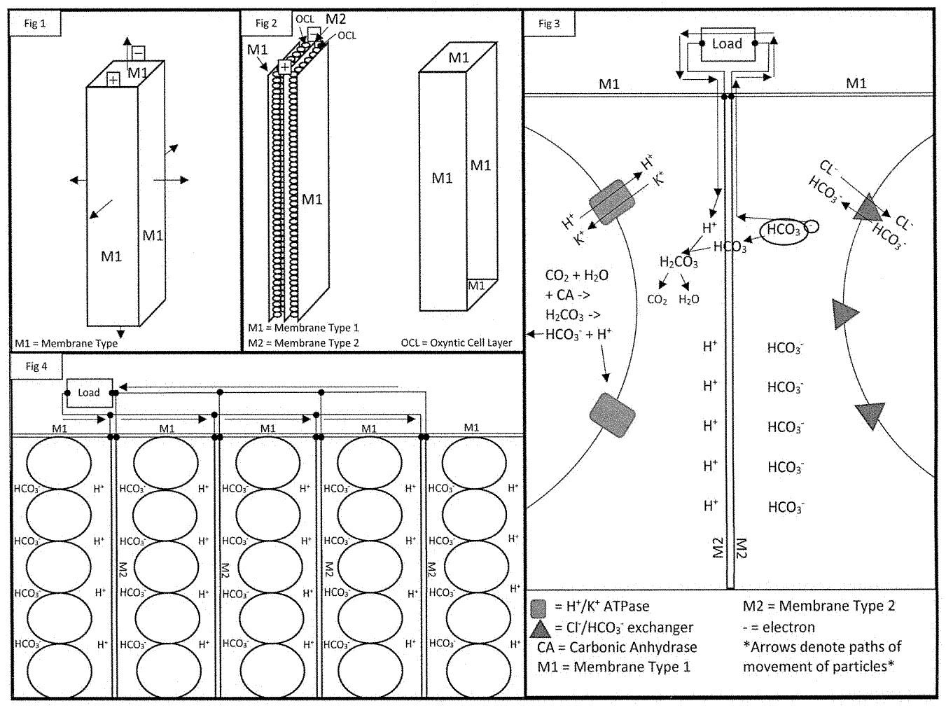

This image shows the outer semipermeable membrane (Membrane Type 1) partially removed to show a simplified, linear biological battery (the equivalent of a single cell in a normal battery). Notice two parallel oxyntic cell layers (OCLs) separated by a membrane permeable to HCO₃ (Membrane Type 2). Membrane Type 2 can be distinguished by the positive and negative leads attached to it. On the outsides of the OCLs lie sections of Membrane Type 1. In reality, all the illustrated sections of Membrane Type 1 would be continuous (as in FIG. 1) and form a barrier to separate the outside cellular environment from the one contained within the battery, allowing nutrients needed for the biological function of the oxyntic cells contained within to diffuse through, but acting as a barrier to proteins like antibodies. Membrane Type 1 also forms continuous perimeter connections with all sections of Membrane Type 2, thus isolating OCLs into a discrete volume known as an OCL compartment. The oxyntic cells share a direction of polarization, which ensures that the ions being pumped to each side of an OCL compartment are of the same kind. In the case of oxyntic cells, this means that H⁺ ions are pumped to one side of the OCL compartment and HCO₃⁻ ions are pumped to the other. Oxyntic cells within the OCL compartments would be packed in tightly enough that they would be flush with adjacent cells and would form cell-to-cell adhesive junctions that divide the OCL compartment into two sub-compartments, thus reducing the mixing of H⁺ and HCO₃⁻ within the OCL compartment as much as possible, which serves to maximize efficiency.

FIG. 3:

Here can be seen a simplified view of two oxyntic cells abutting section of Membrane Type 2, which is capable of converting the energy stored in a conjugate acid-base gradient into electrical energy needed to power a circuit (as described in patent U.S. Pat. No. 4,311,771A). The uniform direction of polarization of OCLs means that the apical sides (which pump H⁺ out of the cell) of oxyntic cells in one chamber abut the basilar sides (which pump HCO₃⁻ out of the cell) of oxyntic cells in a neighboring compartment. Membrane Type 2 separates these two compartments and can convert the ion gradient generated across it into electrical energy. In nature, oxyntic cells, found in stomach epithelium, are polarized so that their basilar side moves HCO₃⁻ into the body via a Cl⁻/HCO₃⁻ exchanger and their apical side pumps H⁺ into the stomach lumen using the protein H⁺/K⁺ ATPase, thus acidifying gastric juices. Oxyntic cells in this battery would have their polarization artificially induced after being placed in compartments. In addition, the oxyntic cells would be immortalized, which would provide a long life for the battery once implanted by ensuring the oxyntic cells which generate the ion gradient that powers it do not die in vivo.

In addition, FIG. 3 also illustrates the biochemical pathway by which HCO₃⁻ and H⁺ are formed from water (H₂O) and carbon dioxide (CO₂) within oxyntic cells. These cells are known to produce a large amount of the enzyme carbonic anhydrase (represented by CA in FIG. 3), which functions to catalyze the conversion of carbon dioxide and water into carbonic acid (H₂CO₃) in a reversible reaction (carbonic anhydrase is incorrectly included as a reagent in the reaction illustrated on the left side of FIG. 3 due to the fact that there was no way to place it above the arrow yet its role is significant enough that it should be included as a part of the reaction). Carbonic acid dissociates into HCO₃⁻ and H⁺ in yet another reversible reaction. The arrows drawn in this illustration are shown to be one way because the internal concentrations of products (HCO₃⁻ and H⁺) are kept low by constant efflux through H⁺/K⁺ ATPase and Cl⁻/HCO₃⁻ exchangers. Thus, the net reaction in oxyntic cells is unidirectional as products are constantly being removed from the cell.

The mechanism by which electrical current is produced using Membrane Type 2 is also illustrated here. An electron dissociates from HCO₃⁻ when it comes in contact with the surface of Membrane Type 2 and subsequently traverses a circuit to reach the opposite surface abutting the high H⁺ concentration. After losing its excess electron, neutral HCO₃ can cross Membrane Type 2 and recombine with H to form carbonic acid (H₂CO₃). Thus, in the region near Membrane Type 2, high concentrations of HCO₃⁻ and H⁺ (able to recombine by passing through Membrane Type 2) drive the formation of H₂CO₃ due to the fact that the two are in equilibrium. Then, this surplus H₂CO₃, due to the fact that it is in equilibrium with CO₂ and H₂O, drives the reaction towards the formation CO₂ and H₂O, dissociating to equilibrium as well. Thus, the net direction of the reaction outside of the oxyntic cells is towards the formation of CO₂ and H₂O, exactly opposite of the same reaction occurring inside of the (biological) cell. Notice that Membrane Type 2 has contacts with an electrical circuit that powers a load. In practice, this would probably be an implanted device of some kind, thus allowing implanted circuitry to be powered by the body's own chemical energy. Oxyntic cells feed off the carbohydrates and fatty acids of the body to create ion gradients whose potential is converted into electrical energy capable of powering a circuit.

FIG. 4:

This illustration shows a simplified circuit diagram of a biological battery comprising 5 OCLs wired in parallel, which is powering an attached electrical device using chemical energy from the body. Carbohydrates and fatty acids that diffuse through the encapsulating ultrafiltration membrane (Membrane Type 1) give the biological cells energy. Amino acids which diffuse through provide the necessary building blocks of cellular machinery. This allows the immortalized oxyntic cells (represented by the oval shapes) to survive and pump ions to different sides of their respective OCL compartments, thus generating a gradient of a conjugate acid-base pair across a membrane (Membrane Type 2) capable of generating electrical energy from the gradient of an acid-base conjugate pair. The electrical energy created from this ion gradient can be put to work powering a circuit which no longer needs an external power source to function within the body of an organism. All the energy is derived internally from the body's chemical energy. Oxyntic cells, being a type of epithelial cell, form cell-cell adhesions, which effectively divide each OCL compartment into two sub-compartments, which reduces the amount of neutralization between HCO₃⁻ and H⁺ that occurs within compartments. It is desirable for efficiency's sake that as much neutralization between conjugate acid-base pairs occur across Membrane Type 2 as possible, because only in this way can energy can be harvested to power a circuit. Note, the arrows in this diagram are used to denote the direction of flow of electrons through the circuit, with electrons traveling from the surface of Membrane Type 2 adjacent the region with a high concentration of HCO₃⁻, into the wire with an arrow pointing towards the load, then into the wire with arrows pointing away from the load and to the surface of Membrane Type 2 adjacent to the region with a high concentration of H⁺. Though the wiring here is in parallel, simple, linear biological batteries can also be made by wiring cells in series as well. In fact, biological batteries can be wired in a combination of parallel and series to modify output voltage and current to suite specific applications.

1. A system capable of generating electrical current from a biologically-generated electrochemical gradient produced by biological cells that can generate an electrochemical gradient across a compartment of space.

2. A process by which electrochemical gradient generating biological cells (ECGGBCs) are encapsulated within a biocompatible membrane permeable to small molecules such as glucose and amino acids but impermeable to proteins which provides them with the chemical material needed to survive and function but prevents the encapsulated ECGGBCs from initiating an immune response.

3. A process by which ECGGBCs are arranged in compartmentalized layers and directionally polarized with uniform orientation in order to generate an electrochemical gradient across sections of membrane which separate adjacent compartments.

4. A process by which encapsulated ECGGBC layers are stacked together and divided from each other by membranes capable of generating electrical energy from an electrochemical gradient across them.

5. A process by which energy stored in an electrochemical gradient generated by ECGGBCs, across membranes capable of generating electrical current from an electrochemical gradient across them, is converted into electrical energy capable of powering an electronic device.
